(19) United States
(12) Patent Application Publication
(10) Pub. No.: US 2012/0213843 A1
(43) Pub. Date: Aug. 23, 2012

(54) COMPOSITION FOR PROMOTING WOUND HEALING

(75) Inventors: Anders Carlsson, Stockholm (SE); Jan Holmback, Vaxholm (SE)

(73) Assignee: Lipidor AB, Stockholm (SE)

(21) Appl. No.: 13/505,502

(22) PCT Filed: Nov. 3, 2010
(86) PCT No.: PCT/SE2010/000269

§ 371 Date: May 2, 2012

(30) Foreign Application Priority Data
Nov. 3, 2009 (SE) 0901408.5
Jan. 12, 2010 (SE) 1000022-2

(51) Int. Cl. A61L 15/34; A61K 38/02; A61P 17/02; A61K 38/16; A61K 38/08; A61K 38/29; A61K 47/44; A61K 38/03

(52) U.S. Cl. 424/445; 514/772; 514/786; 514/777; 514/18.6; 514/9.7; 514/11.8

(57) ABSTRACT

A lipid layer forming wound healing promoting composition comprising volatile silicone oil, polar lipid, C₂-C₄ aliphatic alcohol, and a wound healing agent, in particular a low to medium size natural or synthetic peptide. Also disclosed is a method of forming the lipid layer on a wound and a medical patch provided with the composition.

### FIELD OF THE INVENTION

The present invention relates to a wound healing promoting composition capable of forming a layer comprising a pharmacologically effective amount of a wound healing promoting agent, to a method of manufacture of the composition and to a method of applying it on a wound.

### BACKGROUND OF THE INVENTION

Wound healing promoting agents, in particular peptides of short or intermediate chain length, are known in the art. A few examples are given below.

U.S. Pat. No. 5,015,629 discloses a method of accelerating re-epithelization of wound tissue by applying an amount of the octapeptide angiotensin II to the wound effective for said acceleration.

Subcutaneous injection of parathyroid hormone (1-34) or amino-terminal fragments thereof increases healing of vertebral bone factures in postmenopausal women (R M Neer et al.: Effect of Parathyroid Hormone (1-34) on Fractures and Bone Mineral Density in Postmenopausal Women with Osteoporosis, NEJM 344:19 (2001) 1434-1441).

U.S. Pat. No. 7,452,864 discloses a pharmaceutical composition for topical application to epithelial cells for their regeneration, comprising a novel wound healing peptide of at least 25 amino acids and a bilayer forming lipid carrier comprising a galactolipid, in which the peptide is dispersed.

WO 2008/084253 A1 discloses a pharmaceutical composition comprising a galactolipid material and another active ingredient useful in wound treatment.

WO 01/87344 A1 discloses a pharmaceutical or cosmetic composition comprising one or more pharmaceutically or cosmetically active agent, one or more organosilicon compound based on oligomeric or polymeric diorganosiloxane, and one or more phospholipid. When applied to the skin, the composition of WO 01/87344 A1 penetrates directly within a short period of time into the skin or into the external layers of plants treated with it, so that it cannot be rubbed off since it is rapidly absorbed into the interior of the body. For embodiments intended to be used topically in humans or animals the organosilicon compound of the composition has a boiling point varying between 15° C. and 150° C. at ambient pressure.

The administration of wound healing promoting agents to a wound, in particular a wound in the skin, is however still problematic.

### Objects of the Invention

It is an object of the invention to provide a composition for administration of a wound healing promoting agent, in particular a peptide of short or intermediate length, to a wound of a mammal including man, in which the peptide is present in dissolved form and which is easily applicable to a wound so as to forming a coherent layer on the wound.

It is another object of the invention to provide such a composition that does not irritate the wound.

Still another object of the invention is to provide such a composition that does not cause swelling when applied to the skin.

A further object of the invention is to provide such a composition that does not give a burning feeling when applied to the skin.

Additional objects of the invention will be evident from the following summary of the invention, preferred embodiments thereof described in form of examples, and from the appended claims.

### SUMMARY OF THE INVENTION

According to the present invention is disclosed a composition of the aforementioned kind, comprising or substantially consisting of a polar lipid, a volatile silicone oil, a lower alcohol and a wound healing promoting agent, in particular a peptide of short or intermediate chain length, dissolved in the composition. The composition of the invention consists of a single phase. The low viscosity of the composition allows administration of the composition to a wound by spraying. When sprayed on a wound the composition forms a coherent layer on the wound from which the solvent evaporates or, in respect of the alcohol, is partially absorbed by wound tissue.

The wound healing promoting agent of the invention is a peptide, in particular a short to medium chain length peptide, more preferred a peptide of from six to 120 amino acids, most preferred of from 8 to 45 amino acids. The peptide of the invention can be a naturally occurring peptide or a synthetic peptide. The peptide of the invention can consist of naturally occurring amino acids or comprise naturally occurring amino acids and non-natural amino acids. Preferred wound healing promoting peptides of the invention include angiotensin II, a wound healing fragment, analogue or derivative of angiotensin II, human parathyroid hormone, a wound healing fragment, analogue or derivative of human parathyroid hormone, cathelicidin polypeptide LL37, a wound healing fragment, analogue or derivative of cathelicidin polypeptide LL37. The use of cathelicidin LL37 and derivatives thereof for wound healing is disclosed in U.S. Pat. No. 7,452, 864 incorporated herein by reference.

According to a preferred aspect of the invention, the wound healing promoting agent of the invention is an inhibitor of a pro-inflammatory cytokine, such as one disclosed in U.S. Pat. No. 7,427,589, incorporated herein by reference.

Pro-inflammatory cytokines advantageously blocked according to the invention are, in particular, selected from the group consisting of: tumor necrosis factor (TNF), interleukin 1 (IL-1), interleukin 6 (IL-6), interleukin 8 (IL-8), interleukin 12 (IL-12), interleukin 15 (IL-15), interleukin 17 (IL-17), interleukin 18 (IL-1), granulocytes-macrophage colony stimulating factor (GM-CSF), macrophage colony stimulating factor (M-CSF), monocyte chemotactic protein-1 (MCP-1), macrophage inflammatory protein 1 (MIP-1), RANTES (regulated upon activation, normal T-cell expressed, and presumably secreted), epithelial cell-derived neutrophil attractant-78 (ENA-78), oncostatin-M (OSM), fibroblast growth factor (FGF), platelet derived growth factor (PDGF), and vascular endothelial growth factor (VEGF); and in particular TNF (also called TNF-α) and IL-1 (including both IL-1α and IL-1β).

According to the invention, preferred inhibitors of pro-inflammatory cytokines are: (a) specific TNF blocking agents, such as: monoclonal antibodies, e.g. infliximab, CDP-571 (Humicader™), D2E7, and CDP-870; soluble cytokine receptors, e.g. etanercept, lenercept, pegylated TNF-receptor type I, TBP-1; TNF-receptor antagonists; antisense oligonucleotides, e.g. ISIS-104838; (b) non-specific TNF blocking substances, such as (b1) MMP inhibitors (i.e. matrix metalloproteinase inhibitors, or TACE-inhibitors, i.e., TNF-α Converting Enzyme-inhibitors), tetracyclines, for example doxycycline, lymecycline, oxitetracycline, tetracycline, minocycline and synthetic tetracycline derivatives, such as CMT, i.e., chemically modified tetracyclines; prinomastat (AG3340); batimastat; marimastat; KB-R7785;TIMP-1, TIMP-2, adTIMP-1 (adenoviral delivery of TIMP-1), adTIMP-2 (adenoviral delivery of TIMP-2); (b2) quinolones, for example norfloxacin, levofloxacin, enoxacin, sparfloxacin, temafloxacin, moxifloxacin, gatifloxacin, gemifloxacin, grepafloxacin, trovafloxacin, ofloxacin, ciprofloxacin, pefloxacin, lomefloxacin, temafloxacin; (b3) thalidomide derivates, e.g. SelCID (i.e. Selective Cytokin inhibitors), CC-1088, CDC-501, CDC-801, and linomide (Roquininex®); (b4) lazaroids, e.g., non-glucocorticoid 21-aminosteroids such as U-74389G (16-desmethyl tirilazad) and U-74500; (b4) prostaglandins; iloprost (prostacyclin); (b5) cyclosporine; (b6) pentoxifyllin derivates; (b7) hydroxamic acid derivates; (b8) napthopyrans; (b9) phosphodiesterase I, II, III, IV, and V-inhibitors, e.g., CC-1088, Ro 20-1724, rolipram, amrinone, pimobendan, vesnarinone, SB 207499 (Ariflo®); (b10) melancortin agonists, e.g., HP-228; (c) other TNF blocking substances, such as: (c1) lactoferrin, and peptides derived from lactoferrin such as those disclosed in U.S. Pat. No. 7,253,143 B1, hereby incorporated by reference; (c2) CT3, ITF-2357, PD-168787, CLX-1100, M-PGA, NCS-700, PMS-601, RDP-58, TNF-484A, PCM-4, CBP-1011, SR-31747, AGT-1, solimastat, CH-3697, NR58-3.14.3, RIP-3, Sch-23863, Yissum project no. 11649, Pharma project nos. 6181, 6019 and 4657, SH-636; (d) specific IL-1α and IL-1β blocking substances, such as: monoclonal antibodies, soluble cytokine receptors, IL-1 type II receptor (decoy RII), receptor antagonists; IL-1ra, (Orthogen®, Orthokin®), antisense oligonucleotides; (e) non-specific IL-1α and IL-1β blocking substances, such as: (e1) MMP inhibitors (i.e. matrix metalloproteinase inhibitors); (e2) tetracyclines, for example doxycycline, trovafloxacin, lymecycline, oxitetracycline, tetracycline, minocycline, and synthetic tetracycline derivatives, such as CMT, i.e. chemically modified tetracyclines; (e3) prinomastat (AG3340), batimastat, marimastat, KB-R7785, TIMP-1, TIMP-2, adTIMP-1, adTIMP-2; (e4) quinolones, for example norfloxacin, levofloxacin, enoxacin, sparfloxacin, temafloxacin, moxifloxacin, gatifloxacin, gemifloxacin, grepafloxacin, trovafloxacin, ofloxacin, ciprofloxacin, pefloxacin, lomefloxacin, temafloxacin; (e5) prostaglandins; iloprost (prostacyclin); (e6) cyclosporin; (e7) pentoxyfyllin derivatives; (e8) hydroxamic acid derivatives; (e9) phosphodiesterase I, II, III, IV, and V-inhibitors, CC-1088, Ro 20-1724, rolipram, amrinone, pimobendan, vesnarinone, SB 207499; (f) specific IL-6 blocking substances, such as: (f1) monoclonal antibodies; (f2) soluble cytokine receptors; (f3) receptor antagonists; (f4) antisense oligonucleotides; (g) non-specific IL-6 blocking substances, such as: (g1) MMP inhibitors (i.e. matrix metalloproteinase inhibitors) such as tetracyclines, for example doxycycline, lymecycline, oxitetracycline, tetracycline, minocycline, and synthetic tetracycline derivatives, such as CMT, i.e. chemically modified tetracyclines; prinomastat (AG3340) batimastat, marimastat, KB-R7785, TIMP-1, TIMP-2, adTIMP-1, adTIMP-2; (g2) quinolones, for example norfloxacin, levofloxacin, enoxacin, sparfloxacin, temafloxacin, moxifloxacin, gatifloxacin, gemifloxacin, grepafloxacin, trovafloxacin, ofloxacin, ciprofloxacin, pefloxacin, lomefloxacin, temafloxacin; (g3) prostaglandins; iloprost (prostacyclin); (g4) cyclosporin; (g5) pentoxifyllin derivates; (g6) hydroxamic acid derivates; (g7) phosphodiesterase 1, II, III, IV, and V-inhibitors; CC-11088, Ro 20-1724, rolipram, amrinone, pimobendan, vesnarinone, SB 207499; (g8) melanin and melancortin agonists; HP-228; (h) non-specific IL-8 blocking substances, such as: monoclonal antibodies, soluble cytokine receptors, receptor antagonists, antisense oligonucleotides; (i) non-specific IL-8 blocking substances, such as: (i1) quinolones, for example norfloxacin, levofloxacin, enoxacin, sparfloxacin, temafloxacin, moxifloxacin, gatifloxacin, gemifloxacin, grepafloxacin, trovafloxacin, ofloxacin, ciprofloxacin, pefloxacin, lomefloxacin, temafloxacin; (i2) thalidomide derivates, e.g. SelCID (i.e. Selective Cytokin inhibitors), such as; CC-1088, CDC-501, CDC-801 and linomide (Roquininex®); (i3) lazaroids; (i4) cyclosporine; (i5) pentoxifyllin derivates.

Also useful in the invention are peptides based on the sequence of human lactoferrin disclosed in U.S. Pat. No. 7,253,143 incorporated herein by reference.

According to a second preferred aspect of the invention the wound healing promoting agent is human plasminogen including recombinant varieties thereof and/or another plasma component such as heparin, including a wound healing promoting fragment of heparin.

According to a third preferred aspect of the invention the wound healing agent is a kinin antagonist, in particular a bradikinin antagonist or a kallidin antagonist, such as a kinin antagonist selected from the group consisting of HOE140, NPC17751, NPC349, CP0127, NPC-1776, WIN 64338, des-Arg⁹-bradykinin, des-Arg9-D-Arg-bradykinin and Sar⁴-des-Arg⁹-bradykinin disclosed in U.S. Pat. No. 6,221,845 incorporated herein by reference.

According to a fourth preferred aspect of the invention the wound healing agent is an inhibitor of the interaction between streptococcal M protein, fibrinogen and β2 integrin, such as the tetrapeptide Gly-Pro-Arg-Pro.

According to a fifth preferred aspect of the invention the wound healing agent is a member of the group consisting of: recombinant human 2.5S beta-nerve growth factor disclosed in U.S. Pat. No. 6,063,757 for treating chronic wounds; doxycycline and/or cefaclor disclosed in U.S. 20030092682 A1 for treating cold sores in the mouth, canker sores, cancer wounds, surgical wounds, decubitus ulcers, athletes foot; flavonoid and, optionally, a cinnamic acid derivative disclosed in EP 1300138 A2 for treating eczema, acne, herpes, psoriasis, dermatosis; thrombin-derived peptides disclosed in U.S. Pat. No. 7,049,294 for treating chronic dermal ulcer, such as diabetic ulcer; a pyridine compound disclosed in U.S. 20090069307 A1 for treating skin lesions; a vector encoding hepatocyte growth factor disclosed in U.S. Pat. No. 7,247,620 for treating skin wounds, skin ulcer, bedsore, atopic dermatitis; cyclic guanosine 3′,5′-monophosphate type five phophodiesterase inhibitor disclosed in U.S. 20020065286 A1 for treating non-diabetic chronic wounds and acute wounds; histamine disclosed in U.S. Pat. No. 6,455,565 for treating herpes labialis, cold sores, photodermatitis, thermal burns, pressure sores; xanthine oxidoreductase disclosed in U.S. Pat. No. 6,682,732 for treating skin leasions; ciliary neurotropic factor disclosed in EP 1013280 A1 for treating ulcers including bedsores; erythropoietin disclosed in U.S. 20060166885 A1 for treating decubitus ulcers; N-acyl hydroxyproline disclosed in U.S. 20080188546 A1 for treatment of decubitus ulcers; vascular endothelial growth factor 2 polypeptide or an active fragment thereof disclosed in U.S. 20090192088 A1 for promoting wound healing; an ingenol compound disclosed in U.S. 20090215884 A1 for promoting wound healing; a protein mixture isolated from bone or produced from recombinant proteins comprising growth factor such as bone morphogenic growth factor, the mixture including two or more of BMP-2, PMP-3, BMP-4, BMP-5, BMP-7, TGF-beta 1, TGF beta 2, TGF beta 3, FGF-2 disclosed in U.S. 20060286157 A1 for treating of wounds, for instance diabetic ulcers; human protein 1556A disclosed in U.S. 20080241210 A1 for treating acute wounds, such as lacerations, abrasions, hematoma, and dermatologic diseases; lysophosphotidic acid disclosed in U.S. Pat. No. 6,495,532 for promoting wound healing; substance P disclosed in EP 1658855 A2 for promoting wound healing; the combination of anti-connexin 43 agent and a peptide or protein effective in promoting wound healing, for instance epidermal growth factor, disclosed in U.S. 20090220450 A1 for promoting wound healing; p38 mitogen-activated protein kinase inhibitor disclosed in U.S. 20090170910 A1 for promoting wound healing; trans-glutaminase disclosed in U.S. Pat. No. 5,525,335 promoting wound healing; phenotiazinium compounds disclosed in U.S. 20070161625 A1 for promoting wound healing.

A wound of which the healing can be promoted by the composition of the invention can be, for instance, a shallow or deep wound formed by incision, abrasion or other damage of the skin or a wound caused by thermal burning or scalding of the skin or by chemical burning of the skin, but also a bone fracture or a skin wound caused by bacterial or viral infection or a bedsore. Furthermore, a dermal wound of which the healing can be promoted by the composition of the invention includes irritated, inflamed, burned or mechanically damaged skin. The term “dermal wound” as used in this application thus includes blisters caused by, for instance, bacterial or viral infection or excessive heat. Skin diseased due to eczema, dermatitis and psoriasis is comprised by the term “dermal wound”.

According to a sixth preferred aspect of the invention the wound healing agent is an agent that effectively treats eczema and/or dermatitis and/or psoriasis, such as juniper tar, camphor, menthol, benzocaine, butamben picrate, dibucaine, dibucaine hydrochloride, dimethisoquin hydrochloride, dyclonine hydrochloride, lidocaine, metacresol, lidocaine hydrochloride, pramoxine hydrochloride, tetracaine, tetracaine hydrochloride, benzyl alcohol, camphorated metacresol, phenol, phenolate sodium, resorcinol, diphenhydramine hydrochloride, corticosteroid, such as hydrocortisone and hydrocortisone acetate, and their combinations. Other useful corticosteroids are: tetrahydrocortisol; prednisone; prednisolone; 6α-methylprednisolone; fludrocortisone; 11-desoxycortisol; cortisone; corticosterone; triamcinolone; paramethasone; betamethasone; dexamethasone; desoxycorticosterone acetate; desoxycorticosterone pivalate; fludrocortisone acetate; fuprednisolone; meprednisone; methylprednisolone; methylprednisolone acetate; paramethasone acetate.

The present invention is based on the finding that a particular class of solvents, volatile silicone oils, optionally in combination with a lower aliphatic alcohol, is particularly useful in formulating a composition comprising a polar lipid, suitable for incorporation of a peptide of short or medium length. After application on a wound surface the composition of the invention forms an unstable polar lipid layer from which the volatile silicone oil and, if present, the lower aliphatic alcohol, evaporates readily, leaving a stable oily polar lipid layer substantially consisting of polar lipid comprising the wound healing promoting agent. The low viscosity of the composition of the invention seems, i.a., to be due to the inability of polar lipids to form lyotropic liquid crystals, such as lamellar, hexagonal and various cubic phases of high viscosity. The composition of the invention is clear and of low viscosity even at concentrations of polar lipid as high as 20% by weight.

In contrast, polar lipid compositions corresponding to those of the invention but in which the silicone oil component is substituted by a corresponding weighed amount of water are slightly viscous dispersions at low membrane lipid concentrations or thick gels at 20% membrane lipid by weight of the composition, the highest membrane lipid concentration tested. The high viscosity of the latter composition does not allow it to be administered by spraying. By using the volatile silicone oil of the invention as the diluent instead of water, it is possible to incorporate a surprisingly high amount of polar lipid while only insignificantly affecting viscosity.

Silicone oils of pharmaceutical grade useful in the invention are known in the art. The silicone oils may be either cyclic siloxanes, i.e., cyclomethicones, or short linear siloxanes, i.e., dimethicones. Particularly useful silicone oils include dekamethyl-cyclopentasiloxane (Dow Corning® 345 Fluid) and dodekamethylcyclohexasiloxane (Dow Corning® 246 Fluid). While pentasiloxanes and hexasiloxanes are preferred, tetra-, hepta-, and octasiloxanes are also potentially useful. The silicone oils of the invention can be used in pure form or in admixture.

In addition to chemical inertness the usefulness of silicone oil in the invention is determined by its volatility. In spite of its high boiling point above 180° C., in particular above 200° C., a silicone oil of the invention evaporates easily. This is due to the low heat of vaporization of this class of compounds. In the invention a silicone oil having a heat of vaporization (kJ/kg) at 25° C. of from about 100 kJ/kg to about 300 kJ/kg, more preferred of from about 120 kJ/kg to about 200 kJ/kg are particularly useful. Even more preferred is a silicone oil having a heat of vaporization of from 140 kJ/kg to about 180 kJ/kg at 25° C.

The silicone oil of the invention provides the composition of the invention with at least the following advantageous features: i) the ability to incorporate high contents of polar lipid material; ii) the formation of thermodynamically stable solutions; iii) the low viscosity of the solutions formed making them suitable for, e.g., spraying, dropping, painting or instilling.

The lower aliphatic alcohol of the invention is a C₂ to C₄ alcohol or a mixture of such alcohols, in particular an alcohol selected from C₂ to C₃ alcohol and tert-butanol. Particularly preferred is ethanol.

According to a preferred aspect of the invention, the C₂ to C₄ alcohol may comprise 1,2-propanediol, and/or glycerol, in particular in an amount of up to 5% or 15% by weight of the composition.

The polar lipid of the invention is preferably a membrane lipid such as a phospholipid, a glycolipid, a sphingolipid or a mixture thereof. A particularly preferred phospholipid is phosphatidyl choline. Other preferred phospholipids are phosphatidyl ethanolamine and phosphatidyl inositol. A preferred glycolipid is galactolipid. A preferred galactolipid is digalactosyl-1,2-diacylglycerol as such and in admixture with other galactolipids and/or phospholipids and/or sphingolipids.

Technical scale commercial polar lipids useful in the invention can contain substantial amounts of non-polar lipids, so as to be composed of up to about 50 to 60% by weight of non-polar lipid. Thus, according to a further preferred aspect of the invention, the polar lipid component of the composition of the invention comprises a non-polar lipid in an amount of up to 30% by weight or more, such as up to 50% or 60% by weight and even up to 75% by weight. Preferred non-polar lipids include mono-, di- and triglycerides and their mixtures. A higher content of mono- and diglyceride, in particular of monoglyceride, can be tolerated as a component of the polar lipid of the invention than a corresponding content of triglyceride. The non-polar lipid of the invention can also include fatty acids and their salts, fatty acid esters, fatty acid amides, fatty alcohols, fatty amines, and their mixtures.

The use of a lower aliphatic alcohol such as absolute ethanol for the dissolution of the oily polar lipid of the invention is particularly advantageous with a lipid with a low chain-melting temperature. The chain-melting temperature is the temperature at which the acyl chains of the membrane lipid undergo a phase transition in an excess of water, from a solid-like state to a melted or liquid-like state. Membrane lipid materials like Lipoid S75, Lipoid S45, Phospholipon 50, Lipoid S100, and DOPC all have chain-melting temperatures below 0° C. and can thus be readily dissolved in absolute ethanol at concentrations up to 50% by weight and even higher.

To produce the composition of the invention the polar lipid, in particular a membrane lipid mixture such as lecithin or fractionated oat oil, may alternatively be dissolved in a lower aliphatic alcohol and then diluted with the volatile silicone oil of the invention, resulting in a low-viscous, sprayable, homogenous liquid. Fractionated oat oil is obtained from crude oat oil and is enriched in polar lipids. It typically contains about 50% by weight of non-polar lipids, such as triacylglycerols and diacylglycerols, and about 50% by weight of polar lipids such as phospholipids and glycolipids. Typically, the content of digalactosyldiacylglycerol in a fractionated oat oil is about 20% by weight. Suitable fractionated oat oils are disclosed, for instance, in WO 99/44585 A1.

Lipids like phosphatidyl ethanolamine, particularly dioleylphosphatidyl ethanolamine (DOPE), can also be used as the polar lipid component of the invention as such or in admixture with other polar lipids. DOPE has a chain-melting temperature of −16° C. in water and can be dissolved in absolute ethanol at 50% by weight or higher at elevated temperatures (>60° C.). Such solution can be diluted with volatile silicone oil such as DC 345, resulting in a clear, low-viscous liquid.

Although small amounts of water, such as 1% or 2% and even up to about 5% by weight can be tolerated, the wound healing promoting composition of the invention is preferably substantially water-free, in particular has a water content of less than 5% by weight, preferably of less than 2% or 1% by weight and even less than 0.5% by weight or 0.2% by weight.

According to a preferred aspect, the wound healing promoting composition of the invention comprises from 10% by weight to 30% by weight of membrane lipid, from 10% by weight to 30% by weight of ethanol, from 0.01% by weight to 5% by weight of wound healing agent, the remainder being a volatile silicone oil, with the proviso that the content of volatile silicone oil is 40% by weight or more.

According to another preferred aspect of the invention is disclosed a pharmaceutical carrier composition, that is, a composition of the invention which does not comprise a wound healing promoting agent of the invention but which is suitable for incorporation of such an agent. The carrier composition can comprise from about 30% by weight to about 90% by weight of silicone oil, from about 5% by weight to about 45% by weight of polar lipid, and from about 5% by weight to about 45% by weight of C2 to C4 alcohol, in particular ethanol, optionally 5% by weight or less of water.

According to still another preferred aspect of the invention is disclosed a wound healing promoting agent carrier composition substantially consisting of polar lipid, volatile silicone oil and ethanol in per cent by weight proportions comprised by area F in the phase diagram of FIG. 3, optionally comprising 5% or less by weight of water.

The composition of the invention can be designed to control water loss in a desired manner, that is, from permitting unrestricted or nearly unrestricted water loss to substantially reduced, such as 50% or more per unit time, water loss. The control of water loss is an important factor in wound healing. The control of water loss can be additional to the administration of a wound healing promoting agent by the composition.

The invention will now be described in greater detail by reference to a number of preferred but not limiting examples illustrated in drawing.

### DESCRIPTION OF THE FIGURES

FIG. 1 is a ternary phase diagram illustrating the composition of Example 1;

FIG. 2 is a sectional representation of a medical patch comprising the composition of the invention applied on a shallow skin wound;

FIG. 3 is another ternary phase diagram of lipid layer forming compositions of the invention including carrier compositions for wound healing promoting agents and such compositions comprising wound healing promoting agent;

FIG. 4 is a diagram showing the control of transepidermal water loss by wound healing promoting agent carrier compositions of the invention.

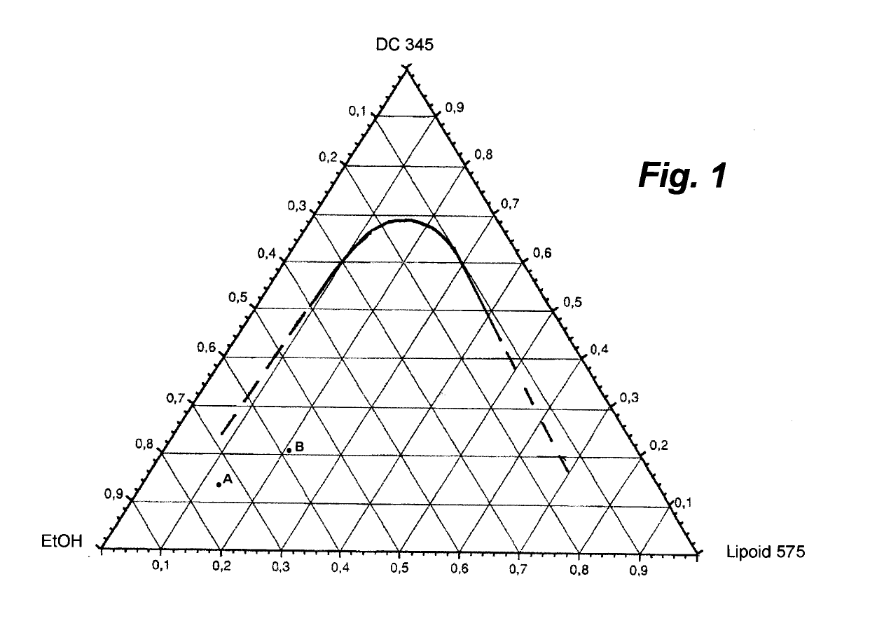

### DESCRIPTION OF PREFERRED EMBODIMENTS

### Materials

TABLE 1
Silicone oils and lipids used in the formulation experiments

| Short name | Supplier, trade name | Chemical name, CAS No. | Lot No. |
|---|---|---|---|
| DC 345 | Dow Corning ® 345 | Dekamethylcyclopentasiloxane, | 5627357 |
| | Fluid | 541-02-6 | |
| DC 245 | Dow Corning ® 245 | Dekamethylcyclopentasiloxane, | 5480964 |
| | Fluid | 541-02-6 | |
| DC 246 | Dow Corning ® 246 | Dodekamethylcyclohexasiloxane, | 5264620 |
| | Fluid | 540-97-6 | |
| DMPC | Lipoid DMPC | Dimyristoylphosphatidylcholine, | 562212-1/13 |
| | | 13699-48-4 | |
| DPPC | Lipoid DPPC | Dipalmitoylphosphatidylcholine, | 563086-1/94 |
| | | 2644-64-6 | |
| DOPC | Lipoid DOPC | Dioleoylphosphatidylcholine, | 566073-1/32 |
| | | 10015-85-7 | |
| DMPG | Lipoid DMPG, Na salt | Dimyristoylphosphatidylglycerol | 602081-1/10 |
| | | sodium salt, 200880-40-6 | |
| DPPG | Lipoid DPPG, Na salt | Dipalmitoylphosphatidylglycerol | 603032-1/36 |
| | | sodium salt, 200880-41-7 | |
| DMPE | Lipoid DMPE | Dimyristoylphosphatidyl- | 699201-1/05 |
| | | ethanolamine, 20255-95-2 | |
| DPPE | Lipoid DPPE | Dipalmitoylphosphatidyl- | 653004-1/19 |
| | | ethanolamine, 3026-45-7 | |
| DOPE | Lipoid DOPE | Dioleoylphosphatidylethanolamine, | 656006- |
| | | 2462-63-7 | 01/012 |
| MOG | Fluka (Sigma-Aldrich), | Monooleoylglycerol, 25496-72-4 | 1384627 |
| | Monoolein | | |
| MCM | Aarhus Karlshamn, | Medium chain monoglycerides | 8192270 |
| | Akoline MCM | | |
| CPL-GL | LTP, CPL ®- | Chromatographically purified | KGL06002 |
| | Galactolipid | galactolipids | |
| O65 | Swedish Oat Fiber, | Galactolipid enriched oat oil | PL 090219 |
| | Oatwell 65 oat oil | | |
| Chol | Sigma-Aldrich, | Cholesterol, 57-88-5 | 057K0683 |
| | Cholesterol | | |
| IPM | Croda, Crodamol IPM | Isopropyl myristate, 110-27-0 | LB03845 |
| S45 | Lipoid S45 | Soy bean lecithin, 8002-43-5 | 745303-1/926 |
| S75 | Lipoid S75 | Soy bean lecithin, 8002-43-5 | 776132- |
| | | | 07/918 |
| S100 | Lipoid S100 | Soy bean lecithin, 8002-43-5 | 790551-7/910 |

Alcohols used in the formulation experiments were ethanol 99.9% (“EtOH”, VWR), 2-propanol HPLC grade (“IPA”, Rathburn), glycerol 99.5% (“Gro”, VWR) and 1,2-propanediol, Ph. Eur. (“PD”, Fluka/Sigma-Aldrich). The materials used in the formulation experiments were provided by the following suppliers: Dow Corning Corp., Midland, Mich., USA; Lipoid GmbH, Ludwigshafen, Germany; Aarhus Karlshamn Sweden AB, Karlshamn, Sweden; LTP Lipid Technologies Provider AB, Karlshamn, Sweden; Swedish Oat Fiber AB, Väröbacka, Sweden; Sigma-Aldrich, St. Louis, Mo., USA; Croda, Goole, East Yorkshire, UK; Rathburn Chemicals Ltd, Walkerburn, Scotland, UK; VWR International AB, Spånga, Sweden; PolyPeptide Laboratories A/S, Hillerød, Denmark; Dermagen AB, Lund, Sweden.

### Wound Healing Promoting Compositions

### EXAMPLE 1

### Formulation of Human Parathyroid Hormone

| | Composition A | Composition B |
|---|---|---|
| Ingredient | % (w/w) | % (w/w) |
| Human PTH (1-34), acetate | 0.05 | 0.05 |
| Phospholipid (Lipoid S75) | 14.0 | 21.1 |
| Absolute ethanol | 14.0 | 21.1 |
| Volatile silicone oil (DC 345) | 71.95 | 57.75 |

To pre-weighed amounts of human parathyroid hormone was added a 50% (w/w) ethanolic phospholipid solution, prepared by dissolving the phospholipid in absolute ethanol at a concentration of 50.0% (w/w). Complete dissolution of the phospholipid was accomplished by short ultrasonication in a bath-type sonicator at about 40° C. and gentle mixing. The resulting clear yellow solutions were diluted with the silicone oil and stored in air-tight glass vials at room temperature.

### EXAMPLE 2

### Formulation of Angiotensin II

| Ingredient | % (w/w) |
|---|---|
| Angiotensin II | 0.05 |
| Phospholipid (Lipoid S75) | 15.0 |
| Absolute ethanol | 15.0 |
| Volatile silicone oil (DC 345) | 69.95 |

To a pre-weighed amount of angiotensin II was added a 50% (w/w) ethanolic phospholipid solution, prepared as described in Example 1. After treatment in a bath-type sonicator at about 35° C., a clear solution was obtained. The solution was diluted with the volatile silicone oil and the resulting clear, light brown to yellow solution was stored in an air-tight glass vial at room temperature.

The appearance of the formulation was unchanged for a month at room temperature. No signs of phase separation or precipitation and subsequent sedimentation were observed, which indicates excellent physical stability.

### EXAMPLE 3

### Formulation of Antimicrobial Cathelicidin Polypeptide LL37

| Ingredient | % (w/w) |
|---|---|
| LL37 Peptide | 0.35 |
| Phospholipid (Lipoid S75) | 11.60 |
| Absolute ethanol | 23.20 |
| Volatile silicone oil (DC 345) | 64.85 |

To a pre-weighed amount of LL37 was added a 33% (w/w) ethanolic phospholipid solution (1:1, by weight) prepared as described in Example 1. After treatment in a bath-type sonicator at about 35° C., a clear solution was obtained. The resulting solution was diluted with the volatile silicone oil. The clear, light brown to yellow solution was stored in an air-tight glass vial at room temperature.

The appearance of the composition was unchanged for more than a month at room temperature, i.e., no signs of phase separation or precipitation and subsequent sedimentation were observed. This indicates excellent physical stability.

### EXAMPLE 4

### Cytokine Inhibitor Wound Healing Composition

| Ingredient | % (w/w) |
|---|---|
| Cyclosporin | 0.46 |
| Phospholipid (Lipoid S75) | 8.59 |
| Absolute ethanol | 21.54 |
| Volatile silicone oil (DC 345) | 69.41 |

To a pre-weighed amount of cyclosporine was added a 28.5% (w/w) ethanolic phospholipid solution. After treatment in a bath-type sonicator at about 35° C., a clear solution was obtained. The resulting solution was diluted with the volatile silicone oil. The clear, light brown to yellow solution was stored in an air-tight glass vial at room temperature. The appearance of the composition was unchanged for more than a month at room temperature.

### EXAMPLE 5

### DPK-060 Wound Healing Promoting Peptide Compositions In Silicone Oil/Lipid Vehicles

Accurately weighed amounts of the peptide DPK-060 were dissolved in mixtures of lipid, glycerol, 1,2-propanediol and ethanol at 40° C. under agitation. Silicone oil

TABLE 2
DPK-060 peptide compositions in silicone oil/lipid vehicles

| | DPK- | | | | | | | | % |
|---|---|---|---|---|---|---|---|---|---|
| | 060 | | % | Gro % | PD % | DC 345 | EtOH % | IPA % | active/ |
| Composition | % w/w | Lipid | w/w | w/w | w/w | % w/w | w/w | w/w | nonvol* |
| KL-DPK-21 | 0.033 | S75 | 3.9 | 6.4 | | 58.2 | 13.0 | 18.5 | 0.32 |
| KL-DPK-22 | 0.199 | S75 | 5.7 | 10.1 | 2.8 | 39.3 | 12.5 | 29.3 | 1.06 |
| KL-DPK-23 | 0.056 | S45 | 3.9 | 6.6 | | 56.8 | 12.5 | 20.1 | 0.53 |
| KL-DPK-24 | 0.129 | S45 | 5.8 | 9.8 | 2.9 | 39.5 | 12.6 | 29.3 | 0.69 |
| KL-DPK-25 | 0.095 | DOPC | 3.8 | 6.6 | | 56.2 | 13.0 | 20.3 | 0.90 |
| KL-DPK-26 | 0.272 | DOPC | 6.8 | 10.3 | 2.8 | 40.8 | 13.1 | 26.0 | 1.34 |
| KL-DPK-27 | 0.036 | O65 | 4.0 | 6.3 | | 54.3 | 11.6 | 23.7 | 0.35 |
| KL-DPK-28 | 0.058 | O65 | 5.6 | 9.6 | 2.8 | 38.9 | 11.4 | 31.5 | 0.32 |
| KL-DPK-29 | 0.096 | DOPE | 4.4 | 6.9 | | 57.8 | 12.9 | 17.9 | 0.84 |
| KL-DPK-31 | 0.125 | DMPC | 4.3 | 6.4 | | 57.1 | 12.7 | 19.3 | 1.15 |
| KL-DPK-40 | 0.167 | S75 | 4.6 | 6.3 | 6.1 | 42.6 | 13.9 | 26.3 | 0.98 |
| KL-DPK-42 | 0.184 | S45 | 5.7 | 10.1 | 2.9 | 40.0 | 11.5 | 29.5 | 0.97 |
| KL-DPK-43 | 0.188 | DOPC | 5.7 | 9.5 | 3.7 | 40.9 | 11.9 | 28.0 | 0.98 |
| KL-DPK-45 | 0.192 | DOPE | 5.9 | 10.3 | 3.1 | 41.7 | 11.8 | 27.1 | 0.99 |
| KL-DPK-47 | 0.189 | DMPC | 5.9 | 10.2 | 3.1 | 40.9 | 11.5 | 28.2 | 0.97 |
| KL-DPK-49 | 0.168 | SM | 4.1 | 6.5 | | 56.0 | 12.7 | 20.6 | 1.57 |
| KL-DPK-50 | — | S75 | 4.7 | 6.2 | 6.1 | 42.8 | 13.7 | 26.4 | — |
| (placebo) | | | | | | | | | |
| KL-DPK-51 | — | DOPE | 4.2 | 6.6 | | 58.9 | 13.2 | 17.1 | — |
| (placebo) | | | | | | | | | |
| KL-DPK-52 | 0.105 | DOPE | 4.0 | 6.6 | | 57.6 | 13.0 | 18.7 | 0.98 |
| KL-DPK-53 | 0.107 | DMPC | 4.2 | 6.6 | | 58.3 | 13.0 | 17.7 | 0.97 |

*Concentration of DPK-060 in % w/w of the non-volatile part of the composition (DC345) and isopropanol was added. The mixture was gently agitated at 40° C. until a homogenous, clear and colourless to brownish yellow liquid was obtained. Table 2 presents representative examples of DPK-060 compositions.

### EXAMPLE 6

### LL-37 Peptide Compositions In Silicone Oil/Lipid Vehicles

Accurately weighed amounts of the peptide LL-37 were dissolved in mixtures of lipid, glycerol and ethanol at 40° C. under agitation. Silicone oil (DC 345) and isopropanol was added and the mixture was gently agitated at 40° C. until a homogenous, clear and slightly yellow to brownish yellow liquid was obtained. Table 3 presents representative examples of LL-37 compositions.

TABLE 3
LL-37 peptide compositions in silicone oil/lipid vehicles

| | | | | | | | | % |
|---|---|---|---|---|---|---|---|---|
| | LL-37 | | % | Gro | DC 345 | EtOH | IPA % | active/ |
| Composition | % w/w | Lipid | w/w | % w/w | % w/w | % w/w | w/w | nonvol* |
| KL-LL37-1 | 0.202 | S75 | 6.9 | 7.1 | 48.5 | 23.3 | 14.0 | 1.42 |
| KL-LL37-2 | 0.184 | DOPE | 5.3 | 8.0 | 49.3 | 26.2 | 11.1 | 1.37 |

*Concentration of LL-37 in % w/w of the non-volatile part of the composition

### Wound Healing Promoting Agent Carrier Compositions

### EXAMPLE 7

### Phospolipid Based Carrier Compositions

Phospholipid was dissolved in mixtures of DC 345 volatile silicone oil and alcohol. The lipid was accurately weighed and mixed with silicone oil and alcohol. The

TABLE 4a
Carrier compositions based on phosphatidyl cholines

| Composition | Lipid | % w/w | DC 345, % w/w | EtOH, % w/w |
|---|---|---|---|---|
| PC-1 | DMPC | 3.8 | 91.4 | 4.8 |
| PC-2 | DMPC | 7.9 | 82.9 | 9.2 |
| PC-3 | DMPC | 16.5 | 62.6 | 20.9 |
| PC-4 | DMPC | 33.3 | 33.4 | 33.4 |
| PC-5 | DOPC | 23.0 | 57.8 | 19.3 |
| PC-6 | DOPC | 22.4 | 38.8 | 38.8 |
| PC-7 | DPPC | 16.5 | 41.7 | 41.7 |

TABLE 4b
Carrier compositions based on phosphatidyl ethanolamines

| | | % | DC 345, % | EtOH, % | IPA, % |
|---|---|---|---|---|---|
| Composition | Lipid | w/w | w/w | w/w | w/w |
| PE-1 | DOPE | 4.5 | 90.7 | | 4.8 |
| PE-2 | DOPE | 4.6 | 90.6 | 4.9 | |
| PE-3 | DOPE | 7.0 | 83.7 | 9.3 | |
| PE-4 | DOPE | 10.3 | 80.8 | 9.0 | |
| PE-5 | DOPE | 14.9 | 63.8 | 21.3 | |

mixture was gently agitated at 40° C. until a homogenous, clear and colourless or slightly yellow liquid was obtained. Table 4a shows examples of compositions based on phosphatidyl cholines and Table 4b compositions based on phosphatidyl ethanolamines.

### EXAMPLE 8

### Acylglycerol Based Carrier Compositions

Commercially available monoglyceride products are mixtures of monoacyl-, diacyl- and small amounts of triacylglycerols. The acylglycerol products were dissolved in mixtures of DC 345 volatile silicone oil and alcohol. The lipid was accurately weighed and mixed with silicone oil and alcohol. The mixture was gently agitated at 40° C. until a homogenous, clear and colourless liquid was obtained. Table 5 shows examples of compositions based on acylglycerols.

TABLE 5
Carrier compositions based on acylglycerols

| | | % | DC 345, | | |
|---|---|---|---|---|---|
| Composition | Lipid | w/w | % w/w | EtOH, % w/w | IPA, % w/w |
| MG-1 | MCM | 13.6 | 86.4 | | |
| MG-2 | MCM | 9.8 | 87.5 | | 2.7 |
| MG-3 | MCM | 21.6 | 74.5 | 3.9 | |
| MG-4 | MCM | 41.2 | 44.1 | 14.7 | |
| MG-5 | MOG | 4.7 | 92.9 | | 2.5 |
| MG-6 | MOG | 4.6 | 91.7 | 3.7 | |
| MG-7 | MOG | 3.6 | 91.6 | 4.8 | |
| MG-8 | MOG | 9.6 | 81.3 | 9.0 | |
| MG-9 | MOG | 19.0 | 60.7 | 20.2 | |
| MG-10 | MOG | 38.3 | 30.8 | 30.8 | |

### EXAMPLE 9

### Carrier Compositions Based On Cholesterol

Compositions comprising cholesterol were prepared by mixing with DC 345 volatile silicone oil and alcohol. The lipid was accurately weighed and mixed with silicone oil and alcohol. The mixture was gently agitated at 40° C. until a homogenous, clear and colourless liquid was obtained. Table 6 shows examples of compositions based on cholesterol.

TABLE 6
Carrier compositions based on cholesterol

| Composition | Cholesterol, % w/w | DC 345, % w/w | EtOH, % w/w |
|---|---|---|---|
| Chol-1 | 1.4 | 88.8 | 9.9 |
| Chol-2 | 2.1 | 73.4 | 24.5 |
| Chol-3 | 3.0 | 48.5 | 48.5 |

### EXAMPLE 10

### Carrier Compositions Based On Galactolipid Rich Materials

Two examples of galactolipid rich materials were used to prepare mixtures with DC 345 volatile silicone oil and alcohols. The lipid was accurately weighed and mixed with silicone oil and alcohols. The mixture was gently agitated at 40° C. until a homogenous, clear and slightly yellow to brownish yellow liquid was obtained. Table 7 shows examples of compositions based on galactolipid rich lipids.

TABLE 7
Carrier compositions based on galactolipid rich materials

| | | | DC 345, | | IPA, |
|---|---|---|---|---|---|
| Composition | Lipid | % w/w | % w/w | EtOH, % w/w | % w/w |
| GL-1 | CPL-GL | 4.9 | 71.3 | 23.8 | |
| GL-2 | CPL-GL | 36.0 | 32.0 | 32.0 | |
| GL-3 | O65 | 3.3 | 73.4 | 4.7 | 18.7 |

### EXAMPLE 11

### Carrier Compositions Based On Lipid Combinations

The ability to combine lipids with different properties in volatile silicon oil/alcohol mixtures was tested. The lipid materials were accurately weighed and mixed with silicone oil and alcohol. The mixture was gently agitated at 40° C. until a homogenous, clear and colourless or slightly yellow liquid was obtained. Table 8 shows examples of compositions based on various combinations of lipids.

TABLE 8
Carrier compositions based on lipid combinations

| | | | | | DC | EtOH, | IPA, |
|---|---|---|---|---|---|---|---|
| | | % | | % | 345, % | % | % |
| Composition | Lipid 1 | w/w | Lipid 2 | w/w | w/w | w/w | w/w |
| Comb-1 | IPM | 8.9 | DOPC | 8.3 | 78.7 | 4.1 | |
| Comb-2 | IPM | 9.0 | DOPE | 5.2 | 81.5 | 4.3 | |
| Comb-3 | MCM | 6.9 | DOPC | 5.8 | 82.9 | 4.4 | |
| Comb-4 | MOG | 10.3 | DOPC | 0.9 | 85.1 | | 3.7 |
| Comb-5 | MCM | 8.9 | Chol | 1.0 | 79.8 | 10.3 | |

### EXAMPLE 12

### Carrier Compositions Based On Commercially Available Lecithin

Commercially available lecithin products are in mixtures of polar lipids (mainly phospholipids) and non-polar lipids (mainly triglycerides). The materials used in the following examples are all obtained from soy beans and contain phosphatidyl choline as the main polar lipid. The lipid was accurately weighed and mixed with silicone oil and alcohol. The mixture was gently agitated at 40° C. until a homogenous, clear and yellow or brownish yellow liquid was obtained. Table 9 shows examples of compositions based on lecithins.

TABLE 9
Carrier compositions based on lecithin

| | | | DC 345, | | IPA, |
|---|---|---|---|---|---|
| Composition | Lecithin | % w/w | % w/w | EtOH, % w/w | % w/w |
| Lec-1 | S45 | 5.6 | 89.7 | 4.7 | |
| Lec-2 | S45 | 9.9 | 81.1 | 9.0 | |
| Lec-3 | S45 | 30.3 | 52.3 | 17.4 | |
| Lec-4 | S45 | 35.8 | 32.1 | 32.1 | |
| Lec-5 | S75 | 14.8 | 76.5 | 4.0 | 4.7 |
| Lec-6 | S75 | 25.4 | 63.4 | 7.0 | 4.2 |
| Lec-7 | S75 | 16.3 | 75.3 | 8.4 | |
| Lec-8 | S75 | 43.4 | 42.5 | 14.2 | |
| Lec-9 | S75 | 39.3 | 30.4 | 30.4 | |
| Lec-10 | S100 | 13.1 | 65.2 | 21.7 | |
| Lec-11 | S100 | 27.3 | 36.3 | 36.3 | |

### EXAMPLE 16

### Carrier Compositions With Different Silicone Oils

The possibility to use different volatile silicone oils was tested by replacing DC 345 by two other silicone oils, DC 245 and DC 246. The lipid was weighed and mixed with silicone oil and alcohol. The mixture was gently agitated at 40° C. until a homogenous, clear and colourless liquid was obtained. Table 10 shows examples of compositions comprising DC 245 and DC 246.

TABLE 10
Carrier compositions with volatile silicone oils DC 245 and DC 246

| | Silicone | % | | | EtOH, % | IPA, % |
|---|---|---|---|---|---|---|
| Composition | oil | w/w | Lipid | % w/w | w/w | w/w |
| Sil-1 | DC 245 | 81.8 | DOPE | 9.1 | 9.1 | |
| Sil-2 | DC 245 | 88.0 | MCM | 5.1 | | 6.9 |
| Sil-3 | DC 245 | 94.0 | MCM | 2.2 | | 3.8 |
| Sil-4 | DC 246 | 83.3 | DOPE | 7.4 | 9.3 | |

### EXAMPLE 13

### Carrier Compositions Based On Lipids And Small Amounts of Water

The possibility to add small amounts of water to the vehicles of the invention was tested. The lipid was accurately weighed and mixed with silicone oil and alcohol. A small amount of water and optionally isopropanol was added. The mixture was gently agitated at 40° C. until a homogenous, clear and colourless or brownish yellow liquid was obtained. Table 11 shows examples of compositions with small amounts of water.

TABLE 11
Carrier compositions with small amounts of water

| | | % | Water, | DC345, | EtOH, % | IPA, % |
|---|---|---|---|---|---|---|
| Composition | Lipid | w/w | % w/w | % w/w | w/w | w/w |
| Wat-1 | DMPC | 7.0 | 4.7 | 79.5 | 8.8 | |
| Wat-2 | DMPG | 2.3 | 5.3 | 69.4 | 23.1 | |
| Wat-3 | DOPE | 6.8 | 2.5 | 58.1 | 14.9 | 17.7 |
| Wat-4 | S75 | 9.7 | 4.4 | 53.7 | 10.8 | 21.5 |
| Wat-5 | S75 | 5.5 | 2.0 | 72.9 | 8.1 | 11.4 |

### EXAMPLE 14

### Miscibility Test

Presented in Table 12 are data on miscibility of ethanolic phospholipid solutions with either volatile silicone oil or water. The mixtures with a low content of PL/ethanol in the silicone oil had a clear appearance immediately after preparation, but separated within a month at room temperature. On the other hand, the formulation with a concentration of PL/ethanol of 20% was miscible with the volatile silicone oil, did not change in appearance during this time period and can thus be considered to be physically stable.

TABLE 12
Dilution of ethanolic phospholipid (PL; Lipoid S75) solutions with volatile
silicone oil (DC 345) and water, respectively.

| Composition | | Volatile | | | | | Appearance | Appearance |
|---|---|---|---|---|---|---|---|---|
| of EtOH | EtOH | silicone | | Conc. of | Conc. of | Conc. of | directly after | after one |
| solution | solution | oil | Water | PL | ethanol | dilution | dilution | month at RT |
| 75.0% PL | 1.01 g | 1.60 g | — | 29.0% | 9.7% | 61.3% | Opaque | |
| | | | | | | | dispersion, | |
| | | | | | | | clear on | |
| | | | | | | | warming | |
| 75.0% PL | 1.01 g | 2.22 g | — | 23.5% | 7.8% | 68.7% | Opaque | |
| | | | | | | | dispersion, | |
| | | | | | | | clear on | |
| | | | | | | | warming | |
| 50.0% PL | 5.00 g | 7.50 g | — | 20.0% | 20.0% | 60.0% | Clear, low- | Unchanged |
| | | | | | | | viscous light | |
| | | | | | | | brown | |
| | | | | | | | solution | |
| 50.0% PL | 5.00 g | — | 7.52 g | 20.0% | 20.0% | 60.0% | Viscous gel | Unchanged |
| 50.0% PL | 0.50 g + | 7.51 g | — | 2.0% | 38.0% | 60.0% | Clear, low- | |
| | 4.51 g | | | | | | viscous light | |
| | neat | | | | | | yellow, | |
| | EtOH | | | | | | opaque | |
| | | | | | | | solution | |
| 50.0% PL | 0.50 g | 4.51 g | — | 5.0% | 5.0% | 90.0% | Clear, low- | Phase |
| | | | | | | | viscous light | separation |
| | | | | | | | yellow | |
| | | | | | | | solution | |
| 50.0% PL | 0.50 g | — | 4.52 g | 5.0% | 5.0% | 90.0% | Homogeneous | Unchanged |
| | | | | | | | viscous | |
| | | | | | | | dispersion | |
| 33.3% PL | 0.50 g | 4.50 g | — | 3.3% | 6.7% | 90.0% | Clear, low- | Phase |
| | | | | | | | viscous light | separation |
| | | | | | | | yellow | |
| | | | | | | | solution | |
| 33.3% PL | 0.50 g | — | 4.52 g | 3.3% | 6.7% | 90.0% | Homogeneous | Unchanged |
| | | | | | | | dispersion | |

All percentages are by weight.

The phospholipid of Table 12 is Lipoid S75 manufactured by Lipoid GmbH, Ludwigshafen, Germany. This phospholipid material from soybean contains about 68-73% of phosphatidylcholine (PC). Other suitable phospholipid materials are, for example, Lipoid S45, Phospholipon 50, and Lipoid S100, all made from soybean and manufactured by Lipoid GmbH, covering a range of PC content of about 50% up to 100%. Further useful phospholipids are synthetic dimyristoylphosphatidylcholine (DMPC), dioleylphosphatidylcholine (DOPC) and dipalmitoylphosphatidylcholine (DPPC).

### EXAMPLE 15

### Medical Patch

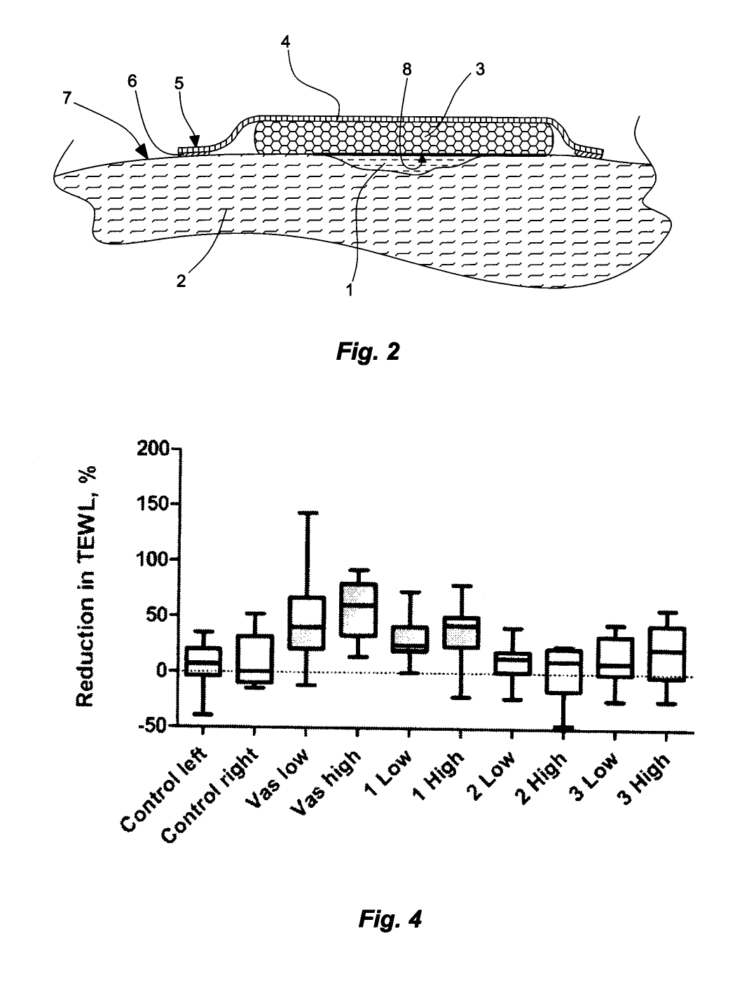

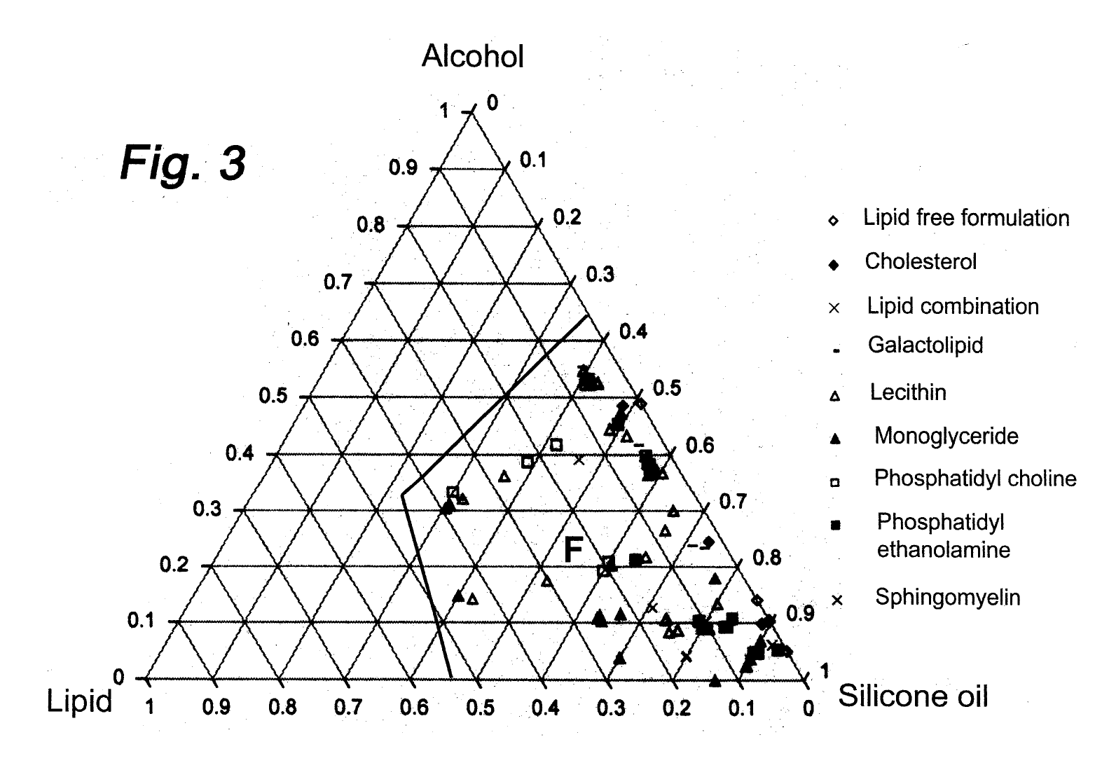

FIG. 2 illustrates schematically a medical patch comprising the composition of Example 3 applied to a shallow skin wound 1 filled with wound serum and coagulated blood. The circular patch (it may be round or square or have any other suitable form), comprises a pad 3 of cotton gauze soaked with the composition of Example 3. The front face of the cotton gauze pad 3 faces the wound 1. The rear face of the gauze layer 3 is attached to a flexible polymer backing 4, which is permeable to silicone oil vapour and alcohol vapour. The backing 4 is of a larger area than the pad 3, which is disposed centered on the backing 4. The periphery 5 of the front face of the backing 4 not covered by the pad 3 is provided with a medical adhesive 6 for attaching the medical patch to the intact skin 7 surrounding the wound 1. Before application the adhesive 6 is protected by a tear-off foil (not shown). After application to the wound 1 a stable polar lipid layer 8 containing LL37 peptide supported by the pad 3 is formed at the boundary between the gauze layer 3 and the wound 1 by evaporation of alcohol and volatile silicone oil. Peptide LL37 leaking from the supported polar lipid layer 8 via wound serum promotes healing of the wound 1. The medical patch of the invention is suitably provided in a sealed polymer container impermeable to solvent vapour and in a sterile state.

### EXAMPLE 16

### Wound Healing Test

A volunteer (male, 66 y) with an incised wound on his left thumb was treated with one drop of the formulation of Example 3. The drop spread easily on the wound surface. The solvent evaporated quickly, leaving a thin lipid layer comprising the wound-healing peptide. The evaporation of the solvent did not give any sense of cooling on the wound and the surrounding skin nor did it cause irritation. The wound healed within two days. According to the volunteer such a wound would otherwise require a considerably longer healing time (of up to two weeks).

### EXAMPLE 17

### Control of Transepidermal Water Loss

Three lipid layer forming compositions of the invention termed A, B, C (Table 13) were tested for their effect on transdermal water loss (TEWL) from a skin surface. Their effect was compared with that of white vaseline (ACO hud, Sweden), a conventional agent for TEWL. The compositions were applied to the skin of ten healthy individuals, 5 women and 5 men; mean age 34 years, SD 18 years, who showed no evidence of skin disease. Prior to application, the volar aspects of their forearms were rapidly cleansed with paper tissue soaked in pure alcohol. Five rectangular areas of 2×2 cm were marked on the volar forearm with a pencil and measured for basal TEWL. The compositions and vaseline were applied to the areas in a randomized manner; one of the areas was left as an untreated control. Two dosages were studied, 3 μl/cm² and 6 μl/cm². Vaseline was used in half of the amount, i.e. 1.5 μl/cm² and 3 μl/cm². The high dose was applied on the right forearm, and the low dose on the left forearm. The products were dispensed onto the surface by means of a displacement micro-pipette (Gilson). The compositions were applied in small droplets onto the area; evaporation was facilitated by slightly blowing at the surface. Vaseline was spread by fingertip.

TABLE 13
Compositions tested for control of transepidermal
water loss (% by weight)

| Composition # | MCM | Polar lipid | EtOH | DC345 |
|---|---|---|---|---|
| 1 | | 15 | 10 | 75 |
| | | (S75) | | |
| 2 | 9 | 1 | 10 | 80 |
| | | (Chol) | | |
| 3 | | 5 | 10 | 85 |
| | | (DOPE) | | |

TEWL was measured before application and 30 min after application by use of DermaLab equipment (open chamber; Cortex Technology, Hadsund, Denmark). The recorded reduction of transepidermal water loss is shown in FIG. 4. The composition 1 of the invention was comparable in effect to Vaseline while compositions 2 and 3 of the invention exerted no significant effect on TEWL.

### EXAMPLE 18

### Bacterial Growth Inhibition Effect of Compositions of the Invention Comprising DPK-060

The growth inhibition effect of compositions KL-DPK-40, -42, -43, -45, -47 and -49 through -53 (Table 2) on cultures of E. coli ATCC 25922, P. aeruginosa ATCC 27853, and S. aureus ATCC 29213 was studied using a radial diffusion assay. Compositions KL-DPK-43, -45, -47, -49, -52, and -53 showed good growth inhibition effect, in particular KL-DPK-45 and -49, while placebo formulations KL-DPK-50 and -51 did not show any effect.

1-28. (canceled)

29. Lipid layer forming wound healing promoting composition comprising volatile silicone oil having a boiling point above 180° C. and a heat of vaporization at 25° C. of from about 100 kj/kg to about 300 kj/kg, polar lipid, wound healing promoting agent, and optionally C2-C aliphatic alcohol.

30. The composition of claim 29, wherein the polar lipid comprises a membrane lipid.

31. The composition of claim 30, wherein the membrane lipid is selected from the group consisting of phospholipid, glycolipid, sphingolipid, and a mixture of two or more of said members.

32. The composition of claim 29, wherein the C2-C4 aliphatic alcohol is ethanol or a combination of propane-1,2-diol and glycerol.

34. The composition of claim 29, additionally comprising a positive amount of less than 5% by weight of water.

35. The composition of claim 29, wherein the silicone oil is a siloxane.

36. The composition of claim 35, wherein the silicone oil is dekamethylcyclopentasiloxane, dodekamethylcyclohexasiloxane or a mixture thereof.

37. The composition of claim 29, wherein the silicone oil has a boiling point above 200° C. and a heat of vaporization at 25° C. of from about 120 kj/kg to about 200 kj/kg.

38. The composition of claim 37, wherein the silicone oil has a heat of vaporization of from 140 kj/kg to 180 kj/kg.

39. The composition of claim 29, wherein the wound healing promoting agent is a peptide.

40. The composition of claim 39, where the peptide is a short to medium chain length peptide having from six to 120 amino acids.

41. The composition of claim 40, wherein the peptide is selected from the group consisting of angiotensin II, a wound healing fragment, analog or derivative of angiotensin II, human arathyroid hormone, a wound healing fragment, analog or derivative of human parathyroid hormone, cathelicidin polypeptide LL37, a wound healing fragment, analog or derivative of cathelicidin polypeptide LL37, and DPK-060.

42. The composition of claim 30, wherein the C2-C4 aliphatic alcohol is present and comprises ethanol, the polar lipid comprises a membrane lipid, the composition contains less than 5% by weight of water, the silicone oil has a boiling point above 200° C. and a heat of vaporization at 25° C. of from about 120 kj/kg to about 200 kj/kg, and the wound healing promoting agent is a short to medium chain length peptide having from six to 120 amino acids.

43. A method of forming a stable polar lipid layer comprising a wound healing promoting agent on a wound comprising applying the composition of claim 29 to the wound so as to form a lipid layer thereon, and evaporating the silicone oil and, if present, the C2-C4 aliphatic alcohol, to form a stable polar lipid layer on the wound.

44. The method of claim 43, wherein the C2-C4 aliphatic alcohol is present and comprisses ethanol, the polar lipid comprises a membrane lipid, the composition contains less than 5% by weight of water, the silicone oil has a boiling point above 200° C. and a heat of vaporization at 25° C. of from about 120 kj/kg to about 200 kj/kg, and the wound healing promoting agent is a short to medium chain length peptide having from six to 120 amino acids.

45. The method of claim 43, wherein the amount of composition applied is selected so as to obtain a stable polar lipid layer of from 1 μm to 500 μm thickness.

46. A method of preparing a wound healing promoting composition, comprising dissolving a pharmacologically effective amount of a wound healing promoting agent in a C2 to C4 aliphatic alcohol to form an alcoholic wound healing promoting agent solution; and combining the alcoholic solution with polar lipid and silicone oil having a boiling point above 180° C. and a heat of vaporization at 25° C. of from about 100 kj/kg to about 300 kj/kg.

47. The method of claim 46, wherein the combining comprises ultrasonication at a temperature of from 20° C. to 50° C.

48. A wound dressing impregnated with the wound healing promoting composition of claim 29.

49. The wound dressing of claim 48 comprising a patch impregnated with the wound healing promoting composition and a backing, wherein the backing is sufficiently porous as to allow passage of silicone oil vapor and alcohol vapor.

50. The wound dressing of claim 49 sealed in a vapor tight polymer enclosure.

51. The wound dressing of claim 50, wherein the C2-C4 aliphatic alcohol is present and comprises ethanol, the polar lipid comprises a membrane lipid, the composition contains less than 5% by weight of water, the silicone oil has a boiling point above 200° C. and a heat of vaporization at 25° C. of from about 120 kj/kg to about 200 kj/kg, and the wound healing promoting agent is a short to medium chain length peptide having from six to 120 amino acids.
